(19) United States
(12) Patent Application Publication
(10) Pub. No.: US 2010/0151526 A1
(43) Pub. Date: Jun. 17, 2010

(54) Method of Producing Sucrose-6-Acetate by Whole-Cell Biocatalysis

(75) Inventors: Rakesh Ratnam, Karnataka (IN); Sundeep Aurora, Karnataka (IN); P. Subramaniyam, Karnataka (IN)

Correspondence Address: THE NATH LAW GROUP, 112 South West Street, Alexandria, VA, 22314, US

(73) Assignee: PHARMED MEDICARE PVT. LTD., Mumbai (IN)

(21) Appl. No.: 11/992,235

(22) PCT Filed: Sep. 21, 2006
(86) PCT No.: PCT/IN2006/000384

§ 371 Date: Jul. 2, 2008

(30) Foreign Application Priority Data
Sep. 22, 2005 (IN) 1174/MUM/2005

(51) Int. Cl. C12P 19/02; C07H 1/06

(52) U.S. Cl. 435/72; 536/127

(57) ABSTRACT

A process is described which uses whole cell preparations, immobilized or without immobilization, of a microorganism, including Aureobasidium pullulans, capable of forming enzymes of the group of fructosyltransferases, for catalyzing a reaction between sucrose and 6-O-protected glucose for formation of 6-O-protected sucrose, an intermediate in synthesis of trichlorogalactosucrose. 6-O-protected Sucrose is separated from high molecular weight by-products of the reaction having molecular weight of 500 daltons and more by reverse osmosis, and further purified by column chromatography.

### TECHNICAL FIELD

The present invention relates to a novel process and a novel strategy for production of 1′-6′-Dichloro-1′-6′-DIDEOXY-β-Fructofuranasyl-4-chloro-4-deoxy-galactopyranoside (TGS) involving use of whole cell biocatalysis for production of its intermediate sucrose-6-acetate.

### BACKGROUND OF THE INVENTION

Strategies of prior art methods of production of 4,1′,6′ trichlorogalactosucrose (TGS) predominantly involve chlorination of 6-O-protected sucrose by use of Vilsmeier-Haack reagent derived from to chlorinate 6-O-protected Sucrose, to form 6 acetyl 4,1′,6′trichlorogalactosucrose, using various chlorinating agents such as phosphorus oxychloride, oxalyl chloride, phosphorus pentachloride etc, and a tertiary amide such as dimethyl formamide (DMF). After the said chlorination reaction, the reaction mass is neutralized to pH 7.0-7.5 using appropriate alkali hydroxides of calcium, sodium, etc. The pH of the neutralized mass is then further raised to 9.5 or above to deacylate/deacetylate the 6 acetyl 4,1′,6′trichlorogalactosucrose to form 4,1′,6′ trichlorogalactosucrose.

This invention relates to the preparation of a key intermediate, Sucrose-6-acetate for the manufacture of the chlorosugar 4,1′,6′trichlorogalactosucrose by microbial bio-catalysis.

Sucrose-6-acetate is a key intermediate in above scheme of production of TGS. Mufti et al (1983) in U.S. Pat. No. 4,380,476 reported a process in which sucrose-6-acetate is a major product of an acylation reaction of sucrose in pyridine with acetic anhydride at a temperature below −20 degrees celcius. Impurities include other monoacylates and also some higher acylates. This process depended on either isolating and obtaining the desired monoacylate in a pure form from others or chlorinating all these acylates and devising means to separate the TGS from other chlorinated sugars.

A process of production was desired which shall produce sucrose-6-acetate without formation of other monoacylates or higher acylates so that isolation and purification of TGS remain as simple as possible

### SUMMARY OF THE INVENTION

This invention describes a process where biomass, including a whole cell mass, derived from a microorganism capable of producing a fructosyltransferase is used to catalyze transfer of a fructose moiety from a fructosyl disaccharide to an acceptor monosaccharide or an acceptor monosaccharide derivative to produce a fructosyldisaccharide or a derivative of fructosyl disaccharide.

A preferred embodiments of this invention relates to the preparation of a key intermediate, Sucrose-6-acetate for the manufacture of the chlorosugar 4,1′,6′trichlorogalactosucrose by microbial bio-catalysis. This embodiment describes a process for making sucrose-6-acetate and analogues compounds from glucose-6-acetate or respective 6-O-protected glucose, bio-catalyzed by whole cells of Aureobasidium pullulans (de Bary) Arn. The sucrose-6-acetate thus obtained is separated from higher molecular saccharides using membrane filtration and can be used for preparation of halo sugars.

### DETAILED DESCRIPTION OF THE INVENTION

There are different types of fructosyltransferase enzymes produced by a variety of microorganisms. The action of different fructosyltransferases from various sources is described in Enzyme and Microbial Technology, 19, 107-117, 1996.

Levansucrase, an enzyme representative of the group of fructosyltransferase is known to catalyse formation of levan, a polyfructose derivative by repeating a process of splitting glucose-fructose link in sucrose and transferring the fructose to an acceptor sugar. Thus, if that acceptor sugar is sucrose itself, it builds up high molecular weight fructose chain. Work of Hestrin and Avigad, in Biochem. J. 69 (1958) pp. 388-398, indicates that a range of sugars acted, with varying degree of ability, as good fructose-acceptors competing with and inhibiting levan formation. Substituted glucose was seen to be poor acceptors. However, when ratio of fructose donor (i.e. sucrose) to acceptor ration is high, typically in the range of 5:1 to 10:1, and concentration is low, it was shown that substituted glucose can also act as an acceptor. Thus Kunst et al in Eur. J. Biochem. 42, 611-620 (1974), succeeded in using D-glucose 6-phosphate as an acceptor with sucrose using an enzyme derived from a mutant of Bacillus subtilis Marburg strain 168. Similarly patent no. GB2046757B disclosed use as an acceptor of a variety of aldose starting materials with sucrose or raffinose wherein a levansucrase was used derived from a variety of microorganisms which included Actinomyces viscosus and B. subtilis (Strain ATCC 6051, i.e. the Marburg strain). In the patent application, however, the aldose is always an underivatised sugar and the mole ratio of donor to acceptor used is 1:5, presumably in order to minimise chain-forming reactions.

Rathbone et al (1986) in U.S. Pat. No. 4,617,269 have claimed a process to prepare 6-derivatised sucrose derivatives by reacting the corresponding 6-derivatised glucose or galactose with a fructosyl transferase in the presence of sucrose or raffinose or stachyose, with a specific limitation that the fructosyltransferase used in such a process is isolated from a bacteria.

In this invention, whole cell preparation of a microorganism is successfully used for transfer of fructose moiety from sucrose to a glucose-6-ester to produce a sucrose-6-ester, the said microorganism being capable of synthesizing one or more of an enzyme of fructosyltransferase group and whole cells of which are amenable for separation from the reaction mixture by a simple process of separation including filtration, centrifugation and the like. It was found that the yields of conversion were very good even with these crude preparations, improving economy and convenience of the method. In the preferred embodiment, yeast Aureobasidium pullulans (de Bary) Arn. is used as a bio-catalyst for achieving preparation of sucrose-6-acetate by reacting glucose-6-acetate with sucrose. However, any other micro-organism may be used in a process of this invention which shall exhibit same activity and function as Aureobasidium pullulans including but not limited to Aspergillus oryzae, Aspergillus awamori, Aspergillus sydowi, Aureobasidium sp., Aspergillus niger, Penicillium roquefortii, Streptococcus mutans, Penicillium jancezewskii, Sachharomyces, Bacillus subtilis, Erwinia and the like.

Colony characteristics of Aureobasidium pullulans are that it grows rapidly in Malt Extract Agar, appearing smooth, soon covered with a slimy exudate, cream-coloured or pink, later mostly becoming brown or black.

The enzyme from the microorganism Aureobasidium pullulans acts on sucrose in the presence of various kinds of monosaccharides, sugar alcohols, alkyl alcohols, glycosides, oligosaccharides and the like as a receptor to transfer the fructosyl group to the receptor molecule exhibiting a very broad receptor specificity. The enzyme from Aureobasidium pullulans is active in the decomposition of sucrose, neokestose, xylsucrose, raffinose and stachyose The whole cells reaction is susceptible to the inhibitive effect of the ions of silver, mercury, zinc, copper and tin.

The said receptor molecule can be any of the following: D-arabinose, L-fructose, 6-deoxyglucose, 6-O-methylgalactose, glucose-6-acetate, glucose-6-propionate, glucose-6-laurate, mellibiose, galactose, xylose glucose-6-phosphate, glucose-6-glutarate, lactose, galactose-6-acetate, mannose, maltose, 1-thio-glucose, maltrotriose, maltopentaose, D-arabinose, maltohexaose, isomaltose, L-arabinose, ribose, lyxose, gluconic acid, L-rhamnose, 6-O-methylglucose, methyl .alpha.-D-glucoside, xylitol, glycerol and the like. Aureobasidium pullulans (de Bary) Arn. is one of the microorganisms, a yeast, which produces fructosyltransferase (SST) enzyme and is found both intra as well as extracellularly. The enzyme from Aureobasidium culture is highly regiospecific in the fructosyl transfer reaction. In the present invention a fructosyltransferase producing Aureobasidium culture ATCC No. 9348 is used for carrying out the preparation of the sucrose-6-acetate by reacting sucrose with 6-O-Acetylglucose. The other higher molecular saccharides produced are separated from sucrose-6-acetate by molecular separation and chromatographic techniques.

Fructosyltransferase is produced by Aureobasidium pullulans by submerged fermentation using suitable media for 72 hrs. In this invention, the enzyme was not isolated from the organism, and instead whole cells are used to achieve the catalysis. In this invention, the microbial cells are preferably separated from the liquid medium by centrifugation and washed with demineralized water. It is, however, conceivable that the cells be used, after attaining a critical growth stage to produce a biomass sufficient to carry out a transfructosyl reaction, with the residual medium itself without separation as a medium for dissolving the donor as well as acceptor of a transfructosyl reaction and the products of the reaction isolated and purified after the reaction is over.

The microbial cell mass is directly suspended into the reaction medium containing sucrose and glucose-6-acetate in a buffer solution. The ratio of sucrose to glucose-6-acetate preferably taken for the reaction is 2:0.5. The reaction is kept under stirring and the formation of sucrose-6-acetate is monitored by HPLC. Appropriate additives, including, but not limited to, invertase inhibitors further including Conduritol-B-epoxide, trestatin, and the like are added to the reaction to avoid any side reactions which may affect the desired product formation As soon as the appropriate titre value of sucrose-6-acetate is obtained, the stirring is stopped and the reaction mixture filtered to separate the microbial cells.

Then the filtrate containing sucrose-6-acetate and other higher molecular weight saccharides is subjected to molecular separation. Here the molecular weight above 500 daltons is separated using suitable membrane separation systems. The lower molecular saccharides are concentrated. It was found that the purity of sucrose-6-acetate obtained was 60%. Further purification was carried out by chromatography on Silanized silica with water as the mobile phase.

The reaction stated above can be made continuous by maintaining sucrose and glucose-6-acetate ratios constant to keep the reaction in the forward direction. Also the microbial cells separated from the reaction can be re used depending on the activity of the enzyme.

The microbial cell mass can also be immobilized by one of the several methods of immobilization of whole cells known in the prior art. Illustrative method used here is adopted from geri, B., Sassi, G., Specchia, V., Bosco, F. and Marzona, M., Process Biochem., 1991, 21, 331-335.

The purified sucrose-6-acetate is taken for chlorination for the preparation of TGS.

Described in the following are examples, which illustrate working of this invention without limiting the scope of this invention in any manner. Reactants, proportion of reactants used, range of reaction conditions described are only illustrative and the scope extends to their analogous reactants, reaction conditions and reactions of analogous generic nature. In general, any equivalent alternative which is obvious to a person skilled in art of clorinated sucrose production is covered within the scope of this specification. Thus, mention of an acetate covers any equivalent acyl group which can perform the same function, and use of a substituted glucose shall cover any substituted aldose which gives same type of analogous reactions under analogous reaction conditions. Several other adaptations of the embodiments will be easily anticipated by those skilled in this art and they are also included within the scope of this specification. Mention in singular is construed to cover its plural also, unless the context does not permit so, viz: use of “an organic solvent” for extraction covers use of one or more of an organic solvent in succession or in a combination as a mixture.

### Example 1

### Growth of Aureobasidium Cells for Biocatalysis

In an experiment, pure culture of Aureobasidium culture obtained from ATCC No. 9348 was grown in 200 ml shake flasks by inoculating one loop full of the said culture from the slant. The culture medium consisting of optimum level of carbon and nitrogen sources was prepared using “Maida” (refined wheat flour made in India), soy flour, yeast extract, phosphates and chlorides. The culture was grown for a period of 48 hours in a rotary shaker at 200 RPM.

The well-grown cells were transferred to a second stage growth culture and growth was continued for 120 hrs. The broth obtained after 120 hrs was centrifuged at 8000 RPM and the cells were separated. The cells were washed with buffer solution twice to get rid of all media constituents sticking to the cells. The cells were then frozen and freeze dried till further use.

### Example 2

### Conversion of Glucose-6-Acetate to Sucrose-6-Acetate by Aureobasidium Cells

100 g of glucose-6-acetate and 380 g of sucrose was taken for the reaction. The reactants were dissolved in 1.2 L of sodium acetate buffer at pH 6.5-7.0. The solution was kept stirring. 250 g of freeze dried Aureobasidium cells were suspended in the solution and temperature was slightly raised up to 35° C. 0.25 g of trestatin was added to the reaction mass to inhibit the invertase activity.

The formation of sucrose-6-acetate was monitored by HPLC. After a reaction time of 90 hrs, 45 g of sucrose-6-acetate formation was recorded in the reaction mixture. The reaction was further continued till 120 hrs and conversion was achieved up to 45% of the glucose-6-acetate added for conversion.

The reaction contents were filtered to remove the suspended cells and then taken for isolation of sucrose-6-acetate by reverse osmosis separation. The RO membrane separated all the lower molecular weight compounds such as glucose and fructose and the higher molecular weight compounds were retained. Then the retained compounds were again diluted with 1:5 times with water and was subjected to nanofiltration at a molecular weight cut off of 500 daltons, and the permeate was collected which was predominantly sucrose-6-acetate and other compounds within the molecular weight of 350-400 daltons. These compounds were again subjected to RO filtration, to concentrate them to more than 20% concentration. Here the purity of sucrose-6-acetate was about 85% and was then loaded on to silanized silica gel column.

The mobile phase used was acetate buffer at pH 9.0-9.5 and the pure sucrose-6-acetate fractions were separated and taken for further concentration and water removal. After the complete removal of water, the sucrose-6-acetate was taken in DMF and taken for chlorination.

The isolated sucrose-6-acetate was 90% pure taken for the preparation of TGS.

### Example 3

### Conversion of Glucose-6-Acetate to Sucrose-6-Acetate by Aureobasidium Cells Immobilized on Eudragit RL 100 (Copolymer of Acrylic Resin)

Aureobasidium cells were immobilized on Eudragit RL100 by following method.

350 g of Aureobasidium cells separated after centrifugation was entrapped in 350 g of sodium alginate by mixing them and extruding as beads. These beads were then coated with Eudragit RL 100 a copolymer of poly acrylic resin.

100 g of glucose-6-acetate and 380 g of sucrose was taken for the reaction. The reactants were dissolved in 1.2 L of sodium acetate buffer at pH 6.5-7.0. The solution was kept stirring. 175 g of immobilized Aureobasidium cells on Eudragit RL100 was added to the solution and temperature was slightly raised up to 45° C.

The formation of sucrose-6-acetate was monitored by HPLC. After a reaction time of 75 hrs, 52 g of sucrose-6-acetate formation was recorded in the reaction mixture. The reaction was further continued till 100 hrs and the conversion was achieved up to 62 g of sucrose-6-acetate, which was 32% of the starting sucrose.

The reaction contents were filtered to remove the suspended cells and then taken for isolation of sucrose-6-acetate by reverse osmosis separation. The RO membrane separated all the lower molecular weight compounds such as glucose and fructose and the higher molecular weight compounds were retained. Then the retained compounds were again diluted with 1:5 times with water and was subjected to nanofiltration at a molecular weight cut off of 500 daltons, and the permeate was collected which was predominantly sucrose-6-acetate and other compounds within the molecular weight of 350-400 daltons. These compounds were again subjected to RO filtration, to concentrate them to more than 20% concentration. Here the purity of sucrose-6-acetate was about 85% and was then loaded on to silanized silica gel column.

The mobile phase used was acetate buffer at pH 9.0-9.5 and the pure sucrose-6-acetate fractions were separated and taken for further concentration and water removal. After the complete removal of water, the sucrose-6-acetate was taken in DMF and taken for chlorination. The isolated sucrose-6-acetate of 92% purity was taken for the preparation of TGS.

### Example 4

### Chlorination of Sucrose-6-Acetate with a Vilsmeier Reagent to Produce TGS

In a 3 L reaction flask, placed 275 ml of Dimethylformamide and cooled to 0-5° C. then added 158 g of Phosphorous pentachloride slowly under stirring to form a Vilsmeier reagent, maintaining the temperature of the reaction mass below 30° C. the mass is further cooled to below 0° C. and the sucrose-6-acetate prepared from example-1 is added slowly at 0-5° C. Then the reaction mess is heated to 80° C. and held for 1 hour, further heated to 100° C. and held for 6 hours and finally at 110-115° C. and held for 2-3 hours. The progress of the reaction is monitored by HPLC analysis.

Then the reaction mixture is cooled to −5 to −8° C. and a 20% solution of Sodium hydroxide is slowly added so as to bring the pH of the mass to 5.5-6.5. This is done to retain the product in acetate form, which facilitates the partition of the desired product in to organic solvents. The yield obtained by this method was 42.3% of the sucrose-6-acetate input.

### Example 5

### Conversion of Glucose-6-Benzoate to Sucrose-6-Benzoate by Aureobasidium Cells Immobilized on Eudragit RL 100 (Copolymer of Acrylic Resin)

A 3% solution (600 ml) of glucose-6-benzoate was prepared in sodium acetate buffer pH 6.5. This solution was pumped using a peristaltic pump into a column (2 cm dia×8 cm ht) which was packed with 12 g of Eudragit RL 100 containing Aureobasidium cells. The outlet from the column was recycled to the feed flask. The flow rate was maintained at 5 ml/min. 60 g of sucrose was added to the feed flask and was dissolved and was kept under constant stirring.

The reaction was continued for 36 hours with periodic analysis of conversion of glucose-6-benzoate to sucrose-6-benzoate along with other impurities formation. At the end of 36 hours, 1.2 g. of sucrose-6-benzoate was formed and the reaction was stopped. The glucose-6-benzoate estimated in the solution was found to be less than 1% and this was made up to 3% by addition of fresh glucose-6-benzoate. The pH was also adjusted to 6.5 and the reaction was continued. This resulted in again a conversion up to 3.6 g of sucrose-6-benzoate at the end of 72 hrs.

1. A process of production of an ester of a fructosyl disaccharide or its derivative from a fructosyl disaccharide comprising:
a. contacting the said fructosyl disaccharide with corresponding ester of an aldose or corresponding ester of a derivative of an aldose in presence of a biomass of a fructosyltransferase-producing micro-organism to produce the said ester of the said fructosyl disaccharide or its derivative, and
b. isolating the said ester of fructosyl disaccharide or its derivative.

2. A process of claim 1 wherein:
a. the said fructosyl disaccharide derivative comprises one or more of an ester including sucrose-6-acetate, sucrose-6-benzoate, 6-O-methyl sucrose, 6-deoxysucrose, galactosucrose, xylsucrose, sucrose-6-glutarate, sucrose-6-propionate, sucrose-6-laurate, and the like,
b. the said fructosyl disaccharide comprises one or more of sucrose, raffinose or stachyose,
c. the said ester of an aldose or corresponding ester of a derivative of an aldose comprising one or more of glucose, galactose, a glucose-6-acetate, glucose-6-benzoate, sucrose-6-butyrate, sucrose-6-glutarate, sucrose-6-laurate, sucrose-6-propionate, sucrose-6-benzoate and the like,
d. the said isolation of the fructosyl disaccharide is achieved by one or a more a method or a combination of a method of isolation and purification including filtration, Reverse Osmosis, Nanofiltration, column chromatography and the like.

3. A process of claim 2 wherein the said micro-organism comprises one or more of a fructosyltransferase producing microorganism including Aureobasidium pullulans, Aspergillus oryzae, Aspergillus awamori, Aspergillus sydowi, Aureobasidium sp., Aspergillus niger, Penicillium roquefortii, Streptococcus mutans, Penicillium jancezewskii, Sachharomyces, Bacillus subtilis, Erwinia and the like.

4. A process of claim 3 wherein a biomass of whole cells of Arabidopsis pullulans is prepared by:
a. repeatedly subculturing from a pure culture in a liquid medium containing nutrients enough to promote their rapid growth,
b. separating the cells from the medium by one or more of a method of separation, preferably by centrifugation,
c. preferably washing the cells free from the medium,
d. using the whole cell mass for catalysis as such or after a refinement or after preservation step including freeze drying.

5. A process of claim 4 where the biomass of whole cell is used either in free form or immobilized on one or more of a solid support.

6. A process of claim 5 for preparation of 6-O-protected sucrose-, preferably a sucrose-6-acetate or a sucrose-6-benzoate comprising steps of:
a. contacting sucrose and 6-O-protected glucose, preferably a glucose-6-acetate or glucose-6-benzoate, further preferably accompanied by shaking, in presence of a freeze dried biomass of Aureobasidium pullulans in free form or in a form immobilized by a method of immobilization including immobilization in alginate beads coated with Eudragit RL 100, a copolymer of acrylic resin,
b. removal of biomass of cells, free or immobilized, by a method of separation, preferably by filtration, and
c. subjecting the process stream for isolation of 6-O-protected sucrose formed.

7. A process of claim 5 for preparation of a 6-O-protected sucrose, preferably of sucrose-6-acetate or sucrose-6-benzoate, comprising steps of:
a. packing biomass of Aureobasidium pullulans immobilized on a solid support in to a column, and
b. passing repeatedly a solution of sucrose and 6-O-protected glucose to form 6-O-protected sucrose, and
c. separating 6-O-protected sucrose from the process stream.

8. A process of claim 6 comprising,
a. subjecting the said process stream containing 6-O-protected sucrose to Reverse Osmosis to remove one or more of a lower molecular weight component including glucose, fructose and the like to get a retaintate containing 6-O-protected sucrose and impurities,
b. subjecting the said retaintate to nanofiltration, preferably after a dilution of about 1:5, at a molecular weight cut off of 500 daltons to get a permeate predominantly containing 6-O-protected sucrose,
c. subjecting the said permeate containing 6-O-protected sucrose to Reverse Osmosis to concentrate the permeate to about 20% or more concentration, and
d. subjecting the concentrated permeate to further purification and isolation by column chromatography.

9. A process of column chromatography of claim 8 wherein, the said concentrated permeate is:
a. loaded on to a silanized silica gel column, using an alkaline buffer at about pH 9.0-9.5, preferably an acetate buffer, as mobile phase, and
b. separating the sucrose-6-acetate as a pure fraction.

10. A process of claim 7 comprising,
a. subjecting the said process stream containing 6-O-protected sucrose to Reverse Osmosis to remove one or more of a lower molecular weight component including glucose, fructose and the like to get a retaintate containing 6-O-protected sucrose and impurities,
b. subjecting the said retaintate to nanofiltration, preferably after a dilution of about 1:5, at a molecular weight cut off of 500 daltons to get a permeate predominantly containing 6-O-protected sucrose,
c. subjecting the said permeate containing 6-O-protected sucrose to Reverse Osmosis to concentrate the permeate to about 20% or more concentration, and
d. subjecting the concentrated permeate to further purification and isolation by column chromatography.
